(19) United States
(12) Patent Application Publication
(10) Pub. No.: US 2024/0065608 A1
(43) Pub. Date: Feb. 29, 2024

(54) System and Method of Assessing a Patient's Visual Function

(71) Applicant: Cadwell Laboratories, Inc., Kennewick, WA (US)

(72) Inventor: Michal Holub, Block 707 (SG)

(21) Appl. No.: 18/344,702

(22) Filed: Jun. 29, 2023

Related U.S. Application Data
(60) Provisional application No. 63/367,416, filed on Jun. 30, 2022.

(51) Int. Cl. A61B 5/398; A61B 3/00; A61B 5/00; A61N 1/36

(52) U.S. Cl. CPC A61B 5/398; A61B 3/0008; A61B 5/6821; A61N 1/36046

(57) ABSTRACT

A system for assessing a patient's visual function includes a first set of electrodes positioned on the patient's scalp, a head-mounted device having a frame with a left eye element, a right eye element, and a bridge coupling the eye elements, light sources accommodated within each of the eye elements, a second set of electrodes integrated in the head-mounted device, and a computing device. When the computing device triggers the one or more light sources to deliver visual stimuli to at least one of patient's eyes, the first set of electrodes records first data and the second set of electrodes record second data. The data is interpreted to differentiate between actual biological causes for visual defects and technical anomalies causing absent or abnormal data in the vision assessment procedure.

### CROSS-REFERENCE

The present specification relies upon U.S. Patent Provisional Application No. 63/367,416, titled “System and Method of Assessing A Patient's Visual Function” and filed on Jun. 30, 2022, for priority.

### FIELD

The present specification relates generally to visual electrophysiological assessment of a patient's visual function. More specifically, the present specification relates to a system and method of using a head-mounted device to provide visual stimuli to a patient as well as record the patient's ERG responses.

### BACKGROUND

ERG (Electroretinography) and VEP (Visual Evoked Potential) are painless, noninvasive tests that use visual electrophysiology to assess a patient's visual function. VEP measures the electrical activity in the entire vision system of the patient. When light enters an eye, it is converted into electrical energy at the retina that travels through the optic nerve to the visual cortex of the brain which processes vision. VEP tests measure the strength and speed of the signal from the retina to the patient's visual cortex in order to detect mechanical or neural abnormalities related to vision. On the other hand, ERG measures the function of the retina which is the light-sensitive layer at the back of an eye. When light enters the eye, it is converted into electrical energy by light-sensitive cells (photoreceptors) in the retina. ERG tests record how well the cells of the retina are conveying electrical impulses within the eye. This test is performed to evaluate and prevent damage to the patient's visual functionality during neurosurgical procedures that may cause visual impairment in the intraoperative period. The patient remains unconscious during surgery under general anesthesia.

In order to provide visual stimulation to the eyes, the patient wears a pair of goggles that have one or more light emitting diodes (LEDs) mounted thereon. When worn, the LEDs are activated to provide visual stimuli to the patient's eyes. As a result of the visual stimulation, VEP responses are recorded using a first set of electrodes positioned on the patient's scalp and ERG responses are recorded using a second set of electrodes. The purpose of the ERG is to assess if the retina of the eye is actually being stimulated by the light emitted by the LEDs or not. When the retina is stimulated by the light from the goggles, the cells in the retina produce an electrical signal that is measured by the second set of electrodes, producing a response 120 as seen in FIG. 1A. The ERG response is a direct response from the retina and does not require the optical nerve or the visual cortex to properly function for a signal to be present. As long as the retina is exposed to light from the goggles, an electrical signal will be recorded by the ERG electrodes. Therefore, during simultaneous VEP and ERG recording, if the ERG response is unaffected and the VEP response is affected, it is an indication that the pathway between the retina and the visual cortex has been compromised. If both the ERG response and the VEP response are affected, then the changes in the VEP response might be caused by a technical issue resulting in a change of the stimulation of the retina (eye movement, movement of goggles etc.). In other words, the simultaneous recording of an ERG response is used as a quality control of the light stimulation of the eye during VEP recording. Simultaneous ERG and VEP recording allows the clinician to understand if a missing VEP cortex response is due to a pathology (VEP response changed, ERG response present) or is a technical error due to the eye/retina actually not being stimulated (VEP response change, ERG response absent).

Thus, misalignment or dislodgement of the ERG electrodes may hinder the recordation of ERG response. As a result, attenuation of the VEP waveform may be mistaken as surgical manipulation (false positivity). Therefore, there is a need for systems and methods for providing simultaneous VEP and ERG recording that include improved, fail-safe, and quick placement of one or more ERG electrodes relative to a patient's eyes, as well as improved reliability of the placement of the LED goggles configured to be worn by the patient.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification discloses a system for assessing a patient's visual function, comprising: a first plurality of electrodes configured to be positioned on the patient's scalp; a head-mounted device comprising a frame, a left eye element positioned in the frame, a right eye element positioned in the frame, and a bridge connecting portions of the frame containing the left eye element and right eye element, wherein when positioned over the patient's periocular region, the frame is adapted to have a first side facing the patient's eyes and a second side opposing the first side; a first set of one or more light sources positioned within the left eye element or portion of the frame containing the left eye element on the first side; a second set of one or more light sources positioned within the right eye element or portion of the frame containing the right eye element on the first side; a second plurality of electrodes integrally positioned on the head-mounted device; and a computing device operably connected to the first plurality of electrodes, the first set of one or more light sources, the second set of one or more light sources, and the second plurality of electrodes, wherein, when the computing device activates at least one of the first set of one or more light sources or second set of one or more light sources to deliver visual stimuli to at least one of the patient's eyes, the computing device is configured to record at least one of a first set of data from the first plurality of electrodes or record a second set of data from the second plurality of electrodes.

Optionally, the head-mounted device is configured as a pair of goggles.

Optionally, the first set of one or more light sources and the second set of one or more light sources are light emitting diodes.

Optionally, when the frame is positioned over the patient's periocular region, the left eye element and the right eye element respectively extend over and cover the patient's left eye and right eye respectively.

Still optionally, on the second side of the frame each of the left eye element and the right eye element comprises an opaque covering or a semi-opaque covering, and wherein on the first side of the frame each of the left eye element and the right eye element houses the first set of one or more light sources and the second set of one or more light sources respectively.

Optionally, each of the left eye element and the right eye element is surrounded by a compliant material, and wherein the second plurality of electrodes are integrated in the compliant material.

Optionally, each of the second plurality of electrodes is a pre-gelled surface electrode.

Optionally, the first data is indicative of visually evoked potential responses and the second data is indicative of electroretinogram responses.

Optionally, the computing device is configured to acquire and process the first data and second data in order to determine whether an anomaly in the first data is due to an error or is indicative of a pathological condition related to the patient's visual function.

Optionally, the second plurality of electrodes includes a first electrode positioned at a first corner of the left eye element, a second electrode positioned at a second corner of the left eye element, a third electrode positioned at a third corner of the right eye element and a fourth electrode positioned at a fourth corner of the right eye element.

Optionally, upon positioning the frame over the patient's periocular region, the first electrode and second electrode correspond to two corners of the patient's left eye whereas the third and fourth electrodes correspond to two corners of the patient's right eye.

In some embodiments, the present specification is directed toward a method of assessing a patient's visual function, comprising: positioning a first plurality of electrodes on the patient's scalp; positioning a visual stimulation device over the patient's periocular region, wherein the visual stimulation device comprises a frame, a left eye element positioned in or one the frame, a right eye element positioned in or one the frame, and a bridge coupling the left eye element and the right eye element, wherein a first set of one or more light sources is coupled to the left eye element, a second set of one or more light sources is coupled to the right eye element, and a second plurality of electrodes are integrally positioned on the visual stimulation device; triggering at least one of the first set of one or more light sources or the second set of one or more light sources to stimulate at least one of the patient's eyes, wherein the first set of one or more light sources and the second set of one or more light sources are triggered by a computing device that is operably connected to the first plurality of electrodes, the first set of one or more light sources, the second set of one or more light sources, and the second plurality of electrodes; using the computing device, recording first data acquired by the first plurality of electrodes; and using the computing device, recording second data acquired by the second plurality of electrodes.

Optionally, the first data and second data are recorded simultaneously. Optionally, the first data and second data are recorded based, at least in part, on when the computing device triggers at least one of the first set of one or more light sources or the second set of one or more light sources. Optionally, the first data and second data are recorded solely based on when the computing device triggers at least one of the first set of one or more light sources or the second set of one or more light sources.

Optionally, the visual stimulation device is configured as a pair of goggles.

Optionally, the first set of one or more light sources and the second set of one or more light sources are light emitting diodes.

Optionally, when the visual stimulation device is positioned over the patient's periocular region, the left eye element and right eye element respectively extend over and cover the patient's left eye and right eye, respectively.

Optionally, when positioned over the patient's periocular region, the visual stimulation device has a first side facing the patient's eyes and a second side opposing the first side, wherein the second side each of the left eye element and the right eye element comprises an opaque covering or semi-opaque covering and wherein at least one of the first side of the left eye element or first side of the right eye element houses at least one of the first set of one or more light sources or the second set of one or more light sources.

Optionally, each of the left and right eye elements is surrounded by a compliant material, and wherein the second plurality of electrodes are integrated in the compliant material.

Optionally, each of the second plurality of electrodes is a pre-gelled surface electrode.

Optionally, the first data is indicative of visually evoked potential responses and the second data is indicative of electroretinogram responses.

Optionally, the computing device is configured to acquire and process the first data and second data in order to determine whether an anomaly in the first data is due to an error or indicative of a pathological condition related to the patient's visual function.

Optionally, the second plurality of electrodes includes a first electrode positioned at a first corner of the left eye element, a second electrode positioned at a second corner of the left eye element, a third electrode positioned at a third corner of the right eye element and a fourth electrode positioned at a fourth corner of the right eye element.

Optionally, upon positioning the visual stimulation device over the patient's periocular region, the first and second electrodes correspond to two corners of the patient's left eye whereas the third and fourth electrodes correspond to two corners of the patient's right eye.

In some embodiments, the present specification discloses a system for assessing a patient's visual function, comprising: a first plurality of electrodes positioned on the patient's scalp; goggles having a frame that includes a left eye element, a right eye element and a bridge connecting the left and right eye elements, wherein when positioned over the patient's periocular region the goggles have a first side facing the patient's eyes and a second side opposite to the first side; one or more light emitting diodes housed within each of the left and right eye elements; a second plurality of electrodes integrated in the goggles, wherein the second plurality of electrodes includes a first electrode positioned at a first corner of the left eye element, a second electrode positioned at a second corner of the left eye element, a third electrode positioned at a third corner of the right eye element and a fourth electrode positioned at a fourth corner of the right eye element; and a computing device operably connected to the first plurality of electrodes, the one or more light emitting diodes and the second plurality of electrodes, wherein when the computing device triggers the one or more light emitting diodes to deliver visual stimuli to at least one of the patient's eyes, the computing device is configured to record first data acquired by the first plurality of electrodes and record second data acquired by the second plurality of electrodes record second data.

Optionally, the first data and the second data are recorded simultaneously. Optionally, the first data and the second data are recorded based, at least in part, on when the computing device triggers at least one of the first set of one or more light sources or the second set of one or more light sources. Optionally, the first data and the second data are recorded solely based on when the computing device triggers at least one of the first set of one or more light sources or the second set of one or more light sources. Optionally, the first data are indicative of visually evoked potential responses and the second data are indicative of electroretinogram responses.

Optionally, the computing device is configured to acquire and process the first data and the second data in order to determine whether an anomaly in the first data is due to a technical issue or indicative of a pathological condition related to the patient's visual function.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a graph illustrating a typical ERG response;

FIG. 1B illustrates a neurophysiological assessment system, in accordance with some embodiments of the present specification;

FIG. 2A shows a head-mounted device configured to be positioned over the periocular region of a patient's face, in accordance with some embodiments of the present specification;

FIG. 2B shows a left eye element of a head-mounted device along with a plurality of electrodes mounted thereon, in accordance with some embodiments of the present specification;

FIG. 2C shows an eye element including an opaque covering, in accordance with some embodiments of the present specification;

FIG. 3 is a flowchart of a plurality of exemplary steps of a method of assessing a patient's visual function, in accordance with some embodiments of the present specification; and

FIG. 4 shows a plurality of waveforms corresponding to ERG and VEP recordings from a left eye and a right eye of a patient during a neurosurgical procedure, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

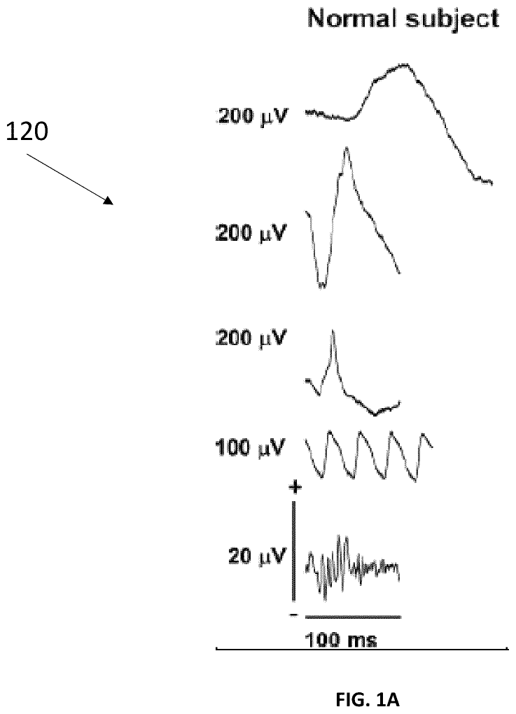

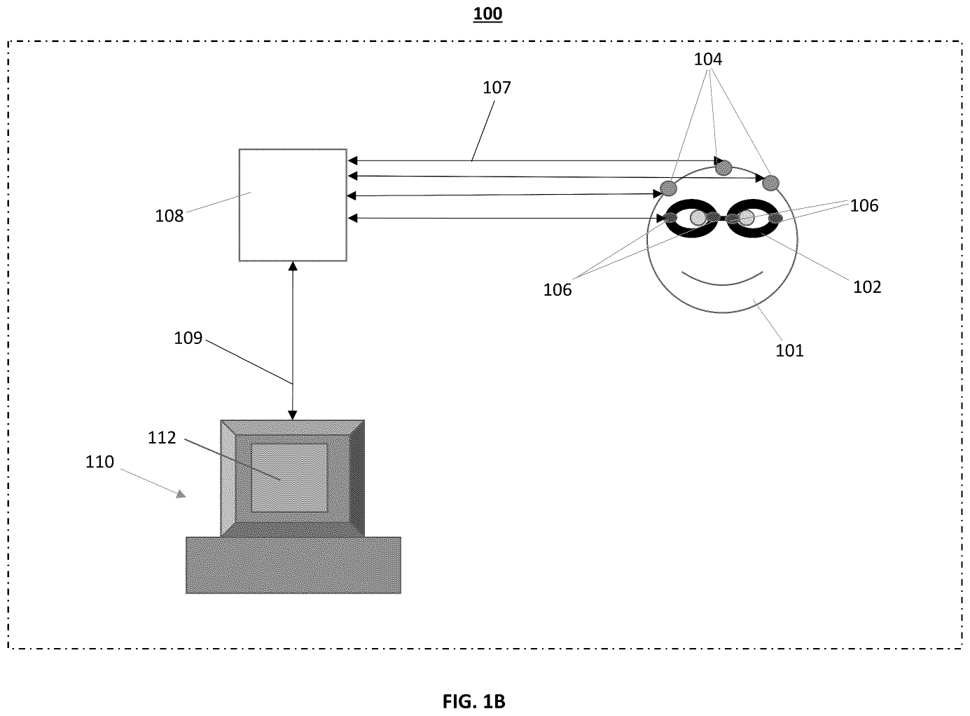

FIG. 1B illustrates a neurophysiological assessment system 100, in accordance with some embodiments of the present specification. In accordance with some aspects, the system 100 is configured to simultaneously generate first data indicative of visual evoked potentials (VEP) and second data indicative of an electroretinogram (ERG) for assessing a visual function of a patient 101. As shown, the system 100 comprises a head-mounted device 102 (also referred to as a “visual stimulation device”) for stimulating the visual system of the patient 101, a first plurality of electrodes 104 positioned on the patient's scalp for recording the first data, a second plurality of electrodes 106 integrally positioned on the head-mounted device 102 for recording the second data, and a computing device 110 operably connected to the head-mounted device 102 as well as the first and second plurality of electrodes 104, 106. It should be appreciated that the first plurality of electrodes 104 may also include a ground reference electrode positioned, for example, on the patient's earlobe. In embodiments, the head-mounted device 102 comprises a pair of goggles configured to be worn by the patient 101.

In some embodiments, the first plurality of electrodes 104 are configured to be positioned over the occipital, parietal, and central scalp in order to record the first data. In some embodiments, signals corresponding to the first and second data may be communicated to an amplifier 108 before being processed by an application program executed on the computing device 110. In embodiments, one or more first wires 107 extend from the first and second plurality of electrodes 104, 106 to the amplifier 108 and one or more second wires 109 extend from the amplifier 108 to the computing device 110, enabling data communication between the first plurality of electrodes 104, second plurality of electrodes 106, the amplifier 108, and the computing device 110. It should be appreciated that, while wired embodiments are discussed herein, all wired connections may be replaced by wireless transmission via one or more transmitters and receivers.

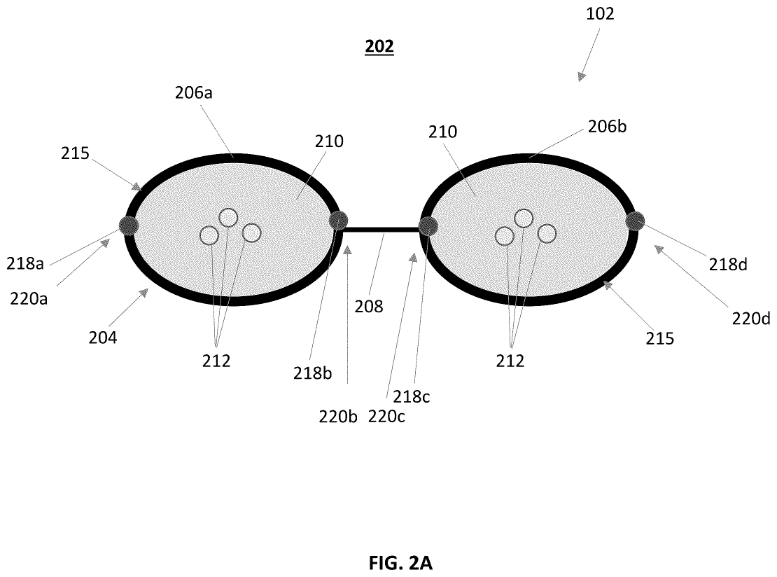

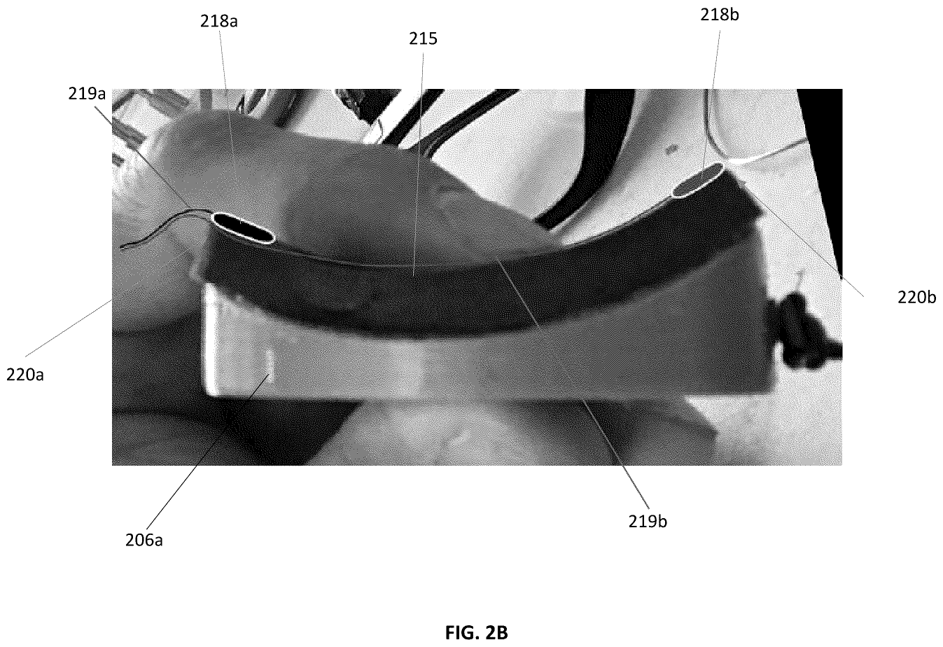

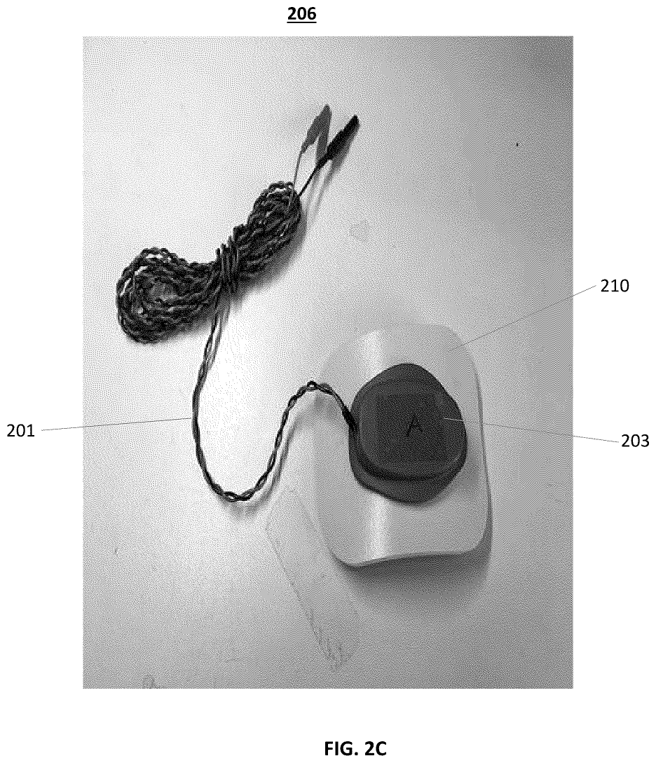

As shown in FIG. 2A, in some embodiments, the head-mounted device 102 is a pair of goggles 202 that are configured to be positioned or accommodated over the periocular region of the patient's face. The goggles 202 provide visual stimuli to the patient. In some embodiments, the goggles 202 are mounted with one or more light sources 212 such as, for example, light emitting diodes (LEDs), to provide flash stimuli to at least one eye of the patient and consequently elicit VEPs that are recorded using the first plurality of electrodes 104 and ERG responses that are recorded using the second plurality of electrodes 106. Typically, VEPs are generated by delivering flashes of light for a period of 10 milliseconds to 500 milliseconds at 1 Hz to 3 Hz by light emitting diodes mounted in the goggles 202. In alternate embodiments, the goggles 202 are virtual reality goggles that are mounted with a video monitor to provide pattern stimuli to the patient.

Referring again to FIG. 2A, the goggles 202 have a frame 204 including a left eye element 206a, a right eye element 206b, and a bridge 208 connecting portions of the frame 204 containing the left eye element 206a and right eye element 206b. Frame 204 has a first side which faces the patient's eyes when the frame 204 is positioned on the patient's periocular region and a second side, opposing the first side, that faces away from the patient. When positioned on the patient's periocular region, the left and right eye elements 206a, 206b respectively extend over and cover the patient's left and right eyes, respectively.

On the second side of the frame 204, each of the left eye element 206a and right eye element 206b includes a covering 210, that ranges from an opaque to semi-opaque or translucent covering or any opacity therebetween. FIG. 2C shows, in accordance with an exemplary embodiment, an eye element 206 that includes a covering 210 that is completely opaque in order to prevent light from entering through the eye element 206. Wires 201, which include one or more light sources are also shown. In an embodiment, wires 201 extend from printed circuit board 203 which is positioned within the covering 210.

Referring back to FIG. 2A, on the first side of the frame 204, in some embodiments, each of the left eye element 206a and right eye element 206b houses a printed circuit board that has the one or more light sources 212 mounted thereon. Stated differently, a first set of the one or more light sources 212 is positioned within the left eye element 206a or portion of the frame 204 containing the left eye element 212 on the first side and a second set of the one or more light sources 212 is positioned within the right eye element 206b or portion of the frame 204 containing the right eye element 206b on the first side. In some embodiments, on the first side of the frame 204, at least one of the left eye element 206a or the right eye element 206b houses at least one of the one or more light sources 212. The patient's visual system is stimulated when the frame 204 is positioned on the patient's periocular region and the one or more light sources 212 are triggered using the computing device 110 (FIG. 1) that is coupled to the frame 204. In embodiments where the one or more light sources 212 are LEDs, the LEDs may all be of a homogenous color/wavelength such as, for example, white or red or may be of heterogeneous colors/wavelengths.

In some embodiments, each of the left eye element 206a and right eye element 206b is surrounded, along the frame 204, by soft compliant material 215 such as, for example, foam padding. The foam padding 215 enables fixation of the goggles 202 to the patient's face around the patient's eyes. In various embodiments, the goggles 202 can be secured to the patient's periocular region using means such as, but not limited to, medical taping, straps, clasps, or elastic bands. Thus, the foam padding 215 helps keep the googles 202 in place so that the goggles do not move around which will change or disable the possibility of stimulating the eye(s).

Referring to FIGS. 1B and 2A, in some embodiments, the second plurality of electrodes 106 includes first electrode 218a, second electrode 218b, third electrode 218c, and fourth electrode 218d mounted in the left eye element 206a and right eye element 206b. In accordance with some aspects of the present specification, the first electrode 218a is positioned at a first corner 220a of the left eye element 206a, the second electrode 218b is positioned at a second corner 220b of the left eye element 206a, the third electrode 218c is positioned at a third corner 220c of the right eye element 206b and the fourth electrode 218d is positioned at a fourth corner 220d of the right eye element 206b. It should be appreciated that the first corner 220a and second corner 220b of the left eye element 206a correspond to the two corners of the patient's left eye while the third corner 220c and fourth corner 220d of the right eye element 206b correspond to the two corners of the patient's right eye.

In some embodiments, each of the first electrode 218a, second electrode 218b, third electrode 218c and fourth electrode 218d is a spherical pre-gelled surface electrode having a diameter of approximately 5 mm. In some embodiments, the first electrode 218a, second electrode 218b, third electrode 218c and fourth electrode 218d are integrated and housed within the frame 204 of the goggles 202. In some embodiments, the first electrode 218a, second electrode 218b, third electrode 218c and fourth electrode 218d are integrated in the foam padding 215 of the goggles 202.

As an example, FIG. 2B shows the left eye element 206a with the first and second electrodes 218a, 218b integrated in the foam padding 215 at the first corner 220a and second corner 220b respectively. In some embodiments, the first electrode 218a and the second electrode 218b are integrated in the foam padding 215 such that the electrodes 218a, 218b are positioned on a surface of the foam padding 215, wherein the surface faces the patient's eye. In embodiments, wires 219a and 219b extend from the first electrode 218a and second electrode 218b, placing the electrodes in data communication with the computing device 110 and amplifier 108. In embodiments, the foam padding 215 assists in keeping the goggles 202 in place on the patient's head by providing a comfortable and secure fit. In embodiments, the goggles 202 are also fixed on the patient's head using additional fastening means on the exterior surface of the goggles. In some embodiments, the goggles 202 are taped onto the patient's head, holding the goggles firmly in position. It should be appreciated that the ERG electrodes integrally positioned on the goggles 202 and the goggles 202 are configured such that they do not move during VEP and ERG recordings, lowering the possibility of changing or disabling stimulation of the patient's eyes.

Referring back to FIG. 2A, since the first electrode 218a, second electrode 218b, third electrode 218c and fourth electrode 218d are pre-installed and integrated in the foam padding 215 (that, in some embodiments, is mounted along the frame 204), during manufacturing of the goggles 202, the positioning of the first electrode 218a, second electrode 218b, third electrode 218c and fourth electrode 218d is predefined and does not need to be managed separately by a clinician. This is in contrast to a conventional setup where the clinician must manually mount the ERG electrodes on a pair of goggles as part of a pre-operative procedure.

When the goggles 202 are mounted on the patient's face, the first electrode 218a, second electrode 218b, third electrode 218c and fourth electrode 218d are, by default, in desired positions with reference to the patient's eyes. The robustness and integrity of the positioning of the first electrode 218a, second electrode 218b, third electrode 218c and fourth electrode 218d lends to a faster application of the goggles 202 and the first electrode 218a, second electrode 218b, third electrode 218c and fourth electrode 218d on the patient's face. Additionally, the wirings/cables, connecting the first electrode 218a, second electrode 218b, third electrode 218c and fourth electrode 218d to the amplifier 108, are simpler to manage and kept physically together. Thus, technical problems associated with false alarms, due to incorrectly positioned or dislodged electrodes, are reduced. Also, keeping the wires/cables physically together leads to improved signal-to-noise ratio and reduced artifacts.

Referring to FIGS. 1B and 2A, when the one or more light sources 212 are triggered using the computing device 110, the visual system of the patient is stimulated resulting in the first plurality of electrodes 104 recording the first data indicative of VEP signals and the second plurality of electrodes 106 recording the second data indicative of ERG signals, simultaneously. In some embodiments, the first data and second data are recorded based, at least in part, on when the computing device triggers at least one of the one or more light sources 212 in the left eye element 206a or the right eye element 206b. In some embodiments, the first data and second data are recorded solely based on when the computing device triggers at least one of the one or more light sources 212 in the left eye element 206a or the right eye element 206b. In embodiments, the first and second data (indicative of VEP and ERG signal, respectively) are displayed to a clinician on a display screen 112 associated with the computing device 110. Thus, the first and second data provide a map of the visual function of at least one eye of the patient. Stated differently, stimulation of the patient's visual system results in recording of neurophysiological signals corresponding to ERG and VEP.

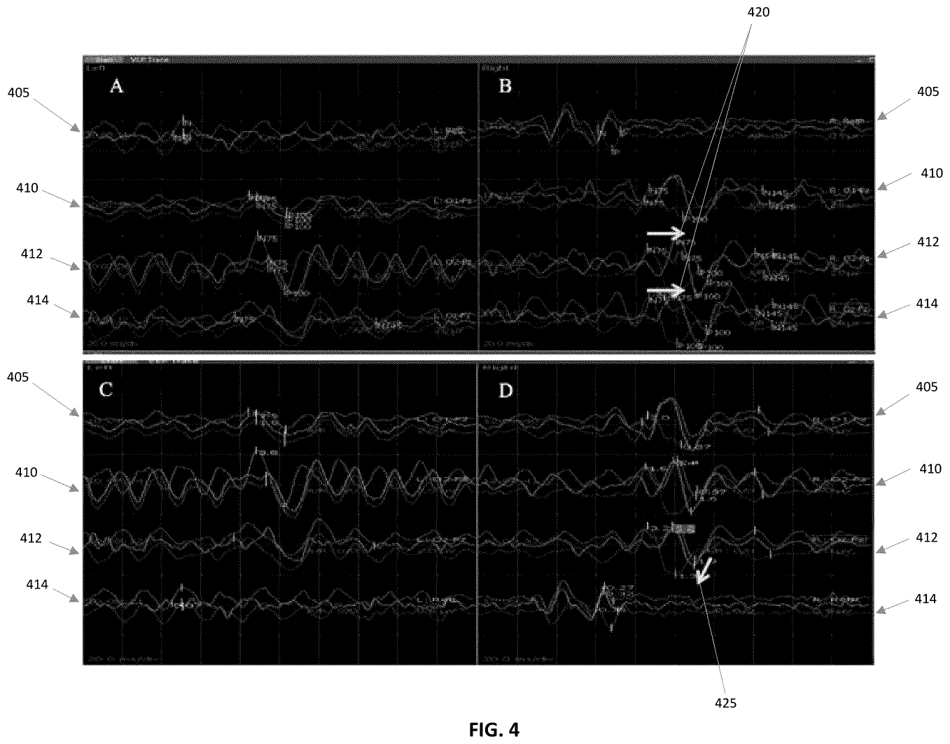

As known to persons of ordinary skill in the art, a) an absence of the first data or VEP response, in the presence of normal second data or a normal ERG response, indicates a compromised function of the optical pathway from the eyes to the occipital lobe or visual cortex of the patient's brain, whereas b) an absence of the first data or VEP response, with an absence of the second data or ERG response, might be caused by an artifact or technical issue resulting in a change of the stimulation of the retina, for example, eye movement or movement of goggles. FIG. 4 shows a plurality of waveforms corresponding to ERG and VEP recordings from a left eye and a right eye of a patient during a neurosurgical procedure, in accordance with some embodiments of the present specification. Segments A and C include ERG and VEP waveforms recorded for the left eye whereas segments B and D include ERG and VEP waveforms recorded for the right eye. For each of the segments A, B, C and D, the first waveform 405 depicts the ERG recording which remains unchanged. Also, for each of the segments A, B, C and D, the second waveform 410, the third waveform 412, and the fourth waveform 414 depict VEP recordings from occipital scalp electrodes (O₁, O₂, O_{z}; International 10-20 System). Segment B, for the right eye, shows a latency shift 420 of the P100 (a positive (downward) deflection on the electrographic tracing that occurs at approximately 100 milliseconds from the time of stimulus presentation) between averages of the baseline and averages of the latest recordings. Segment D, for the right eye, shows an amplitude reduction 425 between averages of the baseline and averages of the latest recordings.

In this context, the recording of the second data (ERG response), simultaneously with the recording of the first data (VEP response), is used as a quality control for the light stimulation of the patient's one or both eyes (visual system). In some embodiments, the computing device 110 is configured to acquire and process the first and second data in order to determine whether an absence or change in the first data is due to a technical anomaly (absence of second data) or indicative of a pathological condition related to the patient's visual function. In some embodiments, first and second maps corresponding respectively to the first and second data are presented on a display screen associated with the computing device 110. Consequently, a clinician observing the display screen can interpret the first and second maps to determine whether an absence or change of the first data is due to a technical anomaly or indicative of a pathological condition related to the patient's visual function. For example, in some embodiments, if the first data (VEP results) is absent, abnormal, or changed and the second data (ERG results) is absent, the clinician may move or re-situate the goggles on the patient's head. If the second data (ERG results) become normal (no longer absent) and the first results (VEP results) also become normal, the clinician will know the initial results were due to technical anomaly (light was not entering patient's retina).

If the second data indicative of the ERG response is present or unaffected (that is, corresponds to that of a typical healthy response) while the first data indicative of the VEP response is absent, anomalistic or affected it is determined that the optical pathway between the retina and the visual cortex is compromised. However, if the second data indicative of the ERG response is absent, anomalistic or affected (that is, does not correspond to that of a typical healthy response) while the first data indicative of the VEP response is also absent, anomalistic or affected (that is, does not correspond to that of a typical healthy response) it is determined that the absent, anomalistic or affected VEP response might be due to a technical anomaly or issue affecting/modifying the stimulation of the patient's retina. The technical anomaly or issue may correspond to at least one of: movement of the patient's eye(s), movement of the head-mounted device 102 or the one or more light sources 212 (in case of the head-mounted device 102 being a pair of goggles with mounted LEDs).

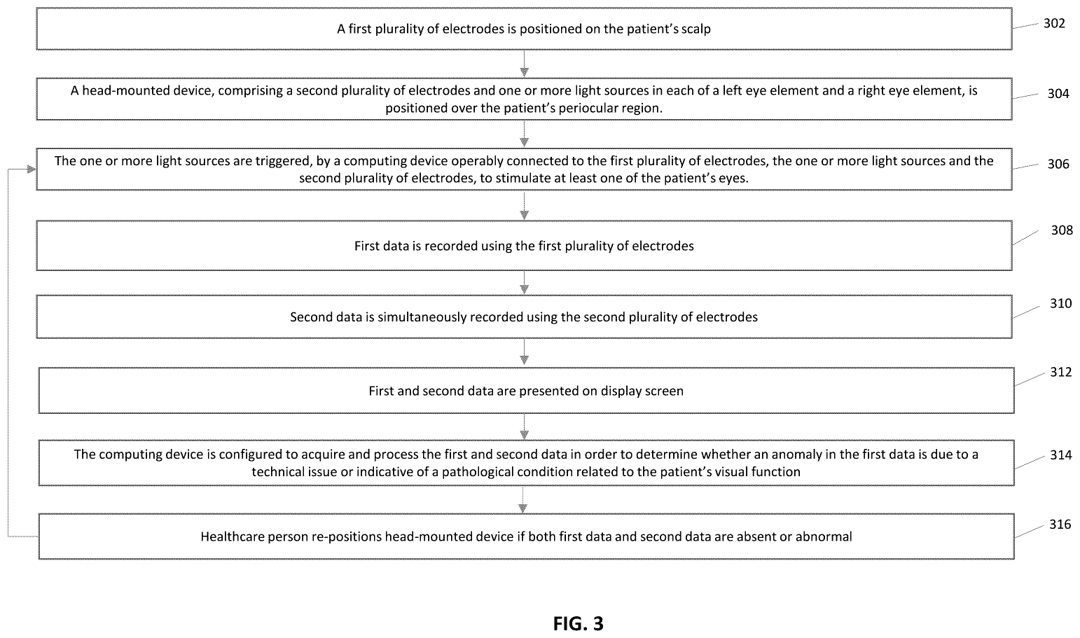

FIG. 3 is a flowchart of a plurality of exemplary steps of a method 300 of assessing a patient's visual function, in accordance with some embodiments of the present specification.

At step 302, a first plurality of electrodes is positioned on the patient's scalp.

At step 304, a head-mounted device (also referred to as a “visual stimulation device”) is positioned over the patient's periocular region. In some embodiments, tape is used to fix the head-mounted device firmly into position. In some embodiments, the head-mounted device has a frame that includes a left eye element, a right eye element and a bridge connecting the left and right eye elements. In some embodiments, one or more light sources are housed in each of the left and right eye elements. In some embodiments, a second plurality of electrodes are integrated in the head-mounted device.

In some embodiments, when positioned over the patient's periocular region, the head-mounted device has a first side facing the patient's eyes and a second side opposite to the first side, wherein on the second side each of the left and right eye elements includes an opaque or semi-opaque covering, and wherein on the first side each of the left and right eye elements houses the one or more light sources.

In some embodiments, each of the left and right eye elements is surrounded by soft compliant material, and wherein the second plurality of electrodes are integrated in the soft compliant material. In some embodiments, the soft compliant material includes foam padding.

In some embodiments, the head-mounted device is a pair of goggles. In some embodiments, the one or more light sources are light emitting diodes. In some embodiments, when the head-mounted device is positioned over the patient's periocular region the left and right eye elements respectively extend over and cover the patient's left and right eyes. In some embodiments, each of the second plurality of electrodes is a pre-gelled surface electrode.

In some embodiments, the second plurality of electrodes includes a first electrode integrally positioned at a first corner of the left eye element, a second electrode integrally positioned at a second corner of the left eye element, a third electrode integrally positioned at a third corner of the right eye element and a fourth electrode integrally positioned at a fourth corner of the right eye element. In some embodiments, upon positioning the head-mounted device over the patient's periocular region, the first and second electrodes correspond to two corners of the patient's left eye whereas the third and fourth electrodes correspond to two corners of the patient's right eye.

At step 306, the one or more light sources are triggered to stimulate at least one of the patient's eyes. In some embodiments, the one or more light sources are triggered by a computing device that is operably connected to the first plurality of electrodes, the one or more light sources and the second plurality of electrodes.

At step 308, first data is recorded using the first plurality of electrodes. In some embodiments, the first data is indicative of visually evoked potential (VEP) responses. At step 310, second data is recorded using the second plurality of electrodes. In embodiments, the first and second data is recorded simultaneously. In some embodiments, the second data is indicative of electroretinogram (ERG) responses.

In some embodiments, the first and second data are presented on a display screen associated with the computing device at step 312.

At step 314, the computing device is configured to acquire and process the first and second data in order to determine whether an anomaly in the first data is due to a technical issue or indicative of a pathological condition related to the patient's visual function. Alternatively, a healthcare person observing the display screen, associated with the computing device, can interpret the displayed first and second data to determine whether an absence of the first data is due to a technical anomaly or indicative of a pathological condition related to the patient's visual function.

At step 316, the healthcare person re-positions the head-mounted device on the patient if both the first data and second data are absent or abnormal. Steps 306 through 314 are then performed again to re-assess the patient's vision function.

The above examples are merely illustrative of the many applications of the system and method of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system for assessing a patient's visual function, comprising:
a first plurality of electrodes configured to be positioned on the patient's scalp;
a head-mounted device comprising a frame, a left eye element positioned in the frame, a right eye element positioned in the frame, and a bridge connecting portions of the frame containing the left eye element and right eye element, wherein when positioned over the patient's periocular region, the frame is adapted to have a first side facing the patient's eyes and a second side opposing the first side;
a first set of one or more light sources positioned within the left eye element or portion of the frame containing the left eye element on the first side;
a second set of one or more light sources positioned within the right eye element or portion of the frame containing the right eye element on the first side;
a second plurality of electrodes integrally positioned on the head-mounted device; and
a computing device operably connected to the first plurality of electrodes, the first set of one or more light sources, the second set of one or more light sources, and the second plurality of electrodes, wherein, when the computing device activates at least one of the first set of one or more light sources or second set of one or more light sources to deliver visual stimuli to at least one of the patient's eyes, the computing device is configured to record at least one of a first set of data from the first plurality of electrodes or record a second set of data from the second plurality of electrodes.

2. The system of claim 1, wherein the head-mounted device is configured as a pair of goggles.

3. The system of claim 2, wherein the first set of one or more light sources and the second set of one or more light sources are light emitting diodes.

4. The system of claim 1, wherein when the frame is positioned over the patient's periocular region, the left eye element and the right eye element respectively extend over and cover the patient's left eye and right eye respectively.

5. The system of claim 1, wherein on the second side of the frame each of the left eye element and the right eye element comprises an opaque covering or a semi-opaque covering, and wherein on the first side of the frame each of the left eye element and the right eye element houses the first set of one or more light sources and the second set of one or more light sources respectively.

6. The system of claim 1, wherein each of the left eye element and the right eye element is surrounded by a compliant material, and wherein the second plurality of electrodes are integrated in the compliant material.

7. The system of claim 1, wherein each of the second plurality of electrodes is a pre-gelled surface electrode.

8. The system of claim 1, wherein the first data is indicative of visually evoked potential responses and the second data is indicative of electroretinogram responses.

9. The system of claim 1, wherein the computing device is configured to acquire and process the first data and second data in order to determine whether an anomaly in the first data is due to an error or is indicative of a pathological condition related to the patient's visual function.

10. The system of claim 1, wherein the second plurality of electrodes includes a first electrode positioned at a first corner of the left eye element, a second electrode positioned at a second corner of the left eye element, a third electrode positioned at a third corner of the right eye element and a fourth electrode positioned at a fourth corner of the right eye element.

11. The system of claim 10, wherein upon positioning the frame over the patient's periocular region, the first electrode and second electrode correspond to two corners of the patient's left eye whereas the third and fourth electrodes correspond to two corners of the patient's right eye.

12. A method of assessing a patient's visual function, comprising:
positioning a first plurality of electrodes on the patient's scalp;
positioning a visual stimulation device over the patient's periocular region, wherein the visual stimulation device comprises a frame, a left eye element positioned in or one the frame, a right eye element positioned in or one the frame, and a bridge coupling the left eye element and the right eye element, wherein a first set of one or more light sources is coupled to the left eye element, a second set of one or more light sources is coupled to the right eye element, and a second plurality of electrodes are integrally positioned on the visual stimulation device;
triggering at least one of the first set of one or more light sources or the second set of one or more light sources to stimulate at least one of the patient's eyes, wherein the first set of one or more light sources and the second set of one or more light sources are triggered by a computing device that is operably connected to the first plurality of electrodes, the first set of one or more light sources, the second set of one or more light sources, and the second plurality of electrodes;
using the computing device, recording first data acquired by the first plurality of electrodes; and
using the computing device, recording second data acquired by the second plurality of electrodes.

13. The method of claim 12, wherein the first data and second data are recorded simultaneously.

14. The method of claim 12, wherein the first data and second data are recorded based, at least in part, on when the computing device triggers at least one of the first set of one or more light sources or the second set of one or more light sources.

15. The method of claim 12, wherein the first data and second data are recorded solely based on when the computing device triggers at least one of the first set of one or more light sources or the second set of one or more light sources.

16. The method of claim 12, wherein the visual stimulation device is configured as a pair of goggles.

17. The method of claim 12, wherein the first set of one or more light sources and the second set of one or more light sources are light emitting diodes.

18. The method of claim 12, wherein when the visual stimulation device is positioned over the patient's periocular region, the left eye element and right eye element respectively extend over and cover the patient's left eye and right eye, respectively.

19. The method of claim 12, wherein when positioned over the patient's periocular region, the visual stimulation device has a first side facing the patient's eyes and a second side opposing the first side, wherein the second side each of the left eye element and the right eye element comprises an opaque covering or semi-opaque covering and wherein at least one of the first side of the left eye element or first side of the right eye element houses at least one of the first set of one or more light sources or the second set of one or more light sources.

20. The method of claim 12, wherein each of the left and right eye elements is surrounded by a compliant material, and wherein the second plurality of electrodes are integrated in the compliant material.

21. The method of claim 12, wherein each of the second plurality of electrodes is a pre-gelled surface electrode.

22. The method of claim 12, wherein the first data is indicative of visually evoked potential responses and the second data is indicative of electroretinogram responses.

23. The method of claim 12, wherein the computing device is configured to acquire and process the first data and second data in order to determine whether an anomaly in the first data is due to an error or indicative of a pathological condition related to the patient's visual function.

24. The method of claim 12, wherein the second plurality of electrodes includes a first electrode positioned at a first corner of the left eye element, a second electrode positioned at a second corner of the left eye element, a third electrode positioned at a third corner of the right eye element and a fourth electrode positioned at a fourth corner of the right eye element.

25. The method of claim 24, wherein upon positioning the visual stimulation device over the patient's periocular region, the first and second electrodes correspond to two corners of the patient's left eye whereas the third and fourth electrodes correspond to two corners of the patient's right eye.

26. A system for assessing a patient's visual function, comprising:
a first plurality of electrodes positioned on the patient's scalp;
goggles having a frame that includes a left eye element, a right eye element and a bridge connecting the left and right eye elements, wherein when positioned over the patient's periocular region the goggles have a first side facing the patient's eyes and a second side opposite to the first side;
one or more light emitting diodes housed within each of the left and right eye elements;
a second plurality of electrodes integrated in the goggles, wherein the second plurality of electrodes includes a first electrode positioned at a first corner of the left eye element, a second electrode positioned at a second corner of the left eye element, a third electrode positioned at a third corner of the right eye element and a fourth electrode positioned at a fourth corner of the right eye element; and
a computing device operably connected to the first plurality of electrodes, the one or more light emitting diodes and the second plurality of electrodes, wherein when the computing device triggers the one or more light emitting diodes to deliver visual stimuli to at least one of the patient's eyes, the computing device is configured to record first data acquired by the first plurality of electrodes and record second data acquired by the second plurality of electrodes record second data.

27. The system of claim 26, wherein the first data and the second data are recorded simultaneously.

28. The system of claim 26, wherein the first data and the second data are recorded based, at least in part, on when the computing device triggers at least one of the first set of one or more light sources or the second set of one or more light sources.

29. The system of claim 26, wherein the first data and the second data are recorded solely based on when the computing device triggers at least one of the first set of one or more light sources or the second set of one or more light sources.

30. The system of claim 26, wherein the first data are indicative of visually evoked potential responses and the second data are indicative of electroretinogram responses.

31. The system of claim 26, wherein the computing device is configured to acquire and process the first data and the second data in order to determine whether an anomaly in the first data is due to a technical issue or indicative of a pathological condition related to the patient's visual function.
